(19) United States
(12) Patent Application Publication
(10) Pub. No.: US 2009/0075836 A1
(43) Pub. Date: Mar. 19, 2009

(54) SCREENING FOR MODULATORS OF METALATION PATHWAYS FOR METALLOPROTEINS

(75) Inventors: Herbert H. Thorp, Carrboro, NC (US); Thomas V. O'Halloran, Chicago, IL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignees: Viamet Pharmaceuticals, Inc., Durham, NC (US); Northwestern University, Evanston, IL (US); University of North Carolina at Chapel Hill, Chapel Hill, NC (US)

(21) Appl. No.: 12/090,764

(22) PCT Filed: Oct. 20, 2006
(86) PCT No.: PCT/US06/41349

§ 371 Date: Oct. 13, 2008

Related U.S. Application Data
(60) Provisional application No. 60/729,183, filed on Oct. 20, 2005.

(51) Int. Cl. C40B 30/06; C12Q 1/02; C40B 30/08; C07K 1/00

(52) U.S. Cl. 506/10; 435/29; 506/11; 530/345; 424/130.1

(57) ABSTRACT

The present invention is directed to compositions and methods for screening of metalation pathways for the metalation of metalloproteins.

### FIELD OF THE INVENTION

The present invention is directed to compositions and methods for screening of metalation pathways for the metalation of metalloproteins.

### BACKGROUND OF THE INVENTION

Thirty percent of all known proteins are metalloproteins that interact closely with a metal ion. For example, every known enzymatic class contains metalloproteins. In general, the structure and function of metalloproteins are highly dependent on the presence of the metal ions, and metal ion concentration in cells is highly regulated. Important metal ions include those of iron, zinc, copper, manganese, magnesium, potassium, calcium, molybdenum, vanadium, tungsten and cobalt

Recent work has shown the presence of particular integral membrane proteins that function to pump metal ions into cells that provides exceptional control over intracellular metal ion concentrations. In addition, metalation pathways are being elucidated. For example, recent work has elucidated the presence of several metallochaparone proteins that guide and protect the metal ions through the cytoplasm and transfer the metals to specific partner proteins. In addition, recent work has also shown that the amount of “free” metal ions with cells is very low. See for example Finney et al., Science 30:931 (2003); Outten et al., Science 292:2488 (2001).

From a therapeutic perspective, there are a number of metalloproteins that are associated with disease states or conditions. For example, cardiovascular disease, cancer, erectile dysfunction and glaucoma have all been associated with metalloproteins (ACE, HDAC and farnesyltransferase, PDE-5 and carbonic anhydrase, respectively). In addition, several genetic diseases, including hemochromatosis, Menkes, Wilson and Lou Gerhig's disease, have been correlated to metalation disorders.

Due to the ubiquitous nature of metalloproteins, and their crucial role in many if not all signaling pathways, there is a need to develop modulators of metalloprotein bioactivity through screening metalation pathways.

### BRIEF SUMMARY OF THE INVENTION

In one aspect, the present invention provides methods of screening for alterations in the metalation of a target protein comprising adding a candidate agent to a cell and determining the metalation status of the target protein. The metalation status can be determined by a bioactivity assay of the protein, or by optionally purifying the protein and determining the presence or absence of metal ions associated with the protein.

In another aspect, the invention provides methods of screening for modulation of target metalation pathway proteins comprising contacting a library of candidate agents with a target pathway protein; and determining the effect of an agent on the activity of the pathway protein. The pathway protein may be a metallochaperone protein, a metal transporter protein or a metalloregulatory protein.

In a further aspect, the invention provides compositions and methods for screening for protein-protein interactions with pathway proteins.

In an additional aspect, the invention provides methods of treating a metalloprotein-associated disorder comprising contacting a metalation pathway protein with an inhibitor such that metal binding to the metalloprotein is decreased.

### DETAILED DESCRIPTION OF THE INVENTION

The present invention is directed to compositions and methods of screening for modulation of the activity of metalloproteins (usually referred to herein as “target proteins” or “target metalloproteins”) and the proteins in the metalation pathway (usually referred to herein as “metalation proteins” or “pathway proteins”), which may or may not be metalloproteins themselves. Currently, it appears that metalloproteins correspond to roughly a third of all structurally characterized proteins. These proteins rely on metal ions for bioactivity. In some cases the bioactivity involves catalytic chemistry at the metal center such as the case with the matrix metalloproteins (MMPs), heme proteins and copper oxidases. In other cases, the metal-associated bioactivity involves structural integrity, such as the case of zinc finger proteins. Other types of metal-associated bioactivity involve regulatory signaling processes such as the case with metalloregulatory proteins or metal trafficking, such as the metallochaperones and metal transporters. That is, metal ions may be important for correct structure, that enables bioactivity, rather than having the metal directly participate in the bioactivity, such as is the case for enzymatic activity in superoxide dismutase (SOD), for example. In addition, metalloproteins can contain both one or more bioactive metals as well as structural metals, and these metals can be the same or different. Foror example SOD contains both Zn and Cu metals, but only the copper is required for catalytic disproportionation of the substrate, ie superoxide anion. Thus, the present invention relies on the fact that the presence of metal ions within many metalloproteins are crucial to activity.

Surprisingly, the amount of free metal ion within the cytoplasm of cells is quite low. See Changela, A., Chen, K., Xue, Y., Holschen, J., Outten, C. E., O'Halloran, T. V., Mondragon, A. “Molecular Basis of Selectivity and Zeptomolar Sensitivity by CueR.” Science, 2003, 301, 1383-1387. Finney, L. A., O'Halloran, T. V. “Transition Metal Speciation in the Cell: Insights from the Chemistry of Metal Ion Receptors.” Science, 2003, 300, 931-936. Outten, C. E., O'Halloran, T. V., “Femtomolar Sensitivity of Metalloregulatory Proteins Controlling Zinc Homeostasis.” Science, 2001, 292, 2488-2492. Rae, T. D., Schmidt, P. J., Pufahl, R. A., Culotta, V. C., and O'Halloran, T. V. “Undetectable Free Intracellular Copper: the Requirement of a Copper Chaperone for Superoxide Dismutase.” Science, 1999, 284, 805-808, all of which are expressly incorporated by reference herein.

As such, metalloproteins appear to “compete” with each other for metal ions, and many require particular metallochaperone proteins for metalation. Due to the fact that most, if not effectively all of the metal ions within a cell are associated with proteins, the present invention is directed to methods for screening for modulators, and particularly inhibitors, of metalation; that is, it appears that one or more pathways may be required to allow metalation of any particular metalloprotein, e.g. there is a “metalation pathway” within cells or living organism that allows for the insertion of the correct metal ions into the apoprotein form of the metalloprotein. Proteins within these pathways are generally referred to herein as “pathway proteins” or “metalation pathway proteins”, as distinguished from target metalloproteins. Thus, the present invention allows for screening for agents that directly or indirectly prevent metalation and thus decrease bioactivity of the metalloprotein itself. Direct screening of the apoprotein form of the metalloproteins is described in “Compositions and Methods of Screening Apoproteins”, filed concurrently with the present application on Oct. 20, 2005, U.S. Ser. No. 60/728,840, herein incorporated by reference in its entirety.

Thus the invention provides methods that allow for screening of candidate agents that bind to factors, particularly proteins, that participate in the metalation pathways to prevent metal binding, and thus metal activation, of the bioactivity of a particular target metalloprotein. Thus there are target metalation proteins, which may or may not be metalated themselves, and target metalloproteins, which require one or more metal ions for activity. These methods can be accomplished in several ways. In one aspect, for example when the full metalation pathway is not yet known, the invention provides for cell-based assays to detect the effect of candidate agents on metalation of a target protein. This allows screening of cells exposed to candidate agents followed by the evaluation of the metalation status of the target metalloprotein. Similarly, cell based assays can be based on a desired, altered phenotype (e.g. due to the inhibition of the target protein; for example, cell apoptosis may occur, a lack of bacterial infectivity is seen, etc.). Once the phenotype is seen, follow on assays are done to determine the site of the action of the agent, and/or an evaluation of the metalation status of the target protein.

In another aspect, the invention provides for biochemical assays based on the activity of particular target pathway proteins involved in metalation pathways. In this embodiment, known metalation pathway proteins, such as metallochaperones, intregral membrane metal transport proteins, etc., are tested for modulation of activity, particularly inhibition, upon exposure to candidate agents.

In a further aspect, the invention provides for assays to identify proteins in metalation pathways. In this case, assays that rely on protein-protein interactions are done with either metalation pathway proteins or metalloproteins, to elucidate the proteins of particular metalation pathways. Once identified, additional screens can be done as outlined herein to find modulators.

### Screening for Alterations in Metalation Pathways

Accordingly, in one aspect, the invention provides for cell-based assays to detect the effect of candidate agents on metalation of target proteins. Thus the invention provides for the addition of candidate agents to cells to screen for modulation of metalation.

### Candidate Agents

By “candidate agent” or “candidate drug” as used herein describes any molecule, e.g., proteins including biotherapeutics including antibodies and enzymes, small organic molecules including known drugs and drug candidates, polysaccharides, fatty acids, vaccines, nucleic acids, etc. that can be screened for activity as outlined herein. Candidate agents are evaluated in the present invention for discovering potential therapeutic agents that affect metalloprotein activity and therefore potential disease states, for elucidating toxic effects of agents (e.g. environmental pollutants including industrial chemicals, pesticides, herbicides, etc.), drugs and drug candidates, food additives, cosmetics, etc., as well as for elucidating new pathways associated with agents (e.g. research into the side effects of drugs, etc.).

Candidate agents encompass numerous chemical classes. In one embodiment, the candidate agent is an organic molecule, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

“Known drugs” or “known drug agents” or “already-approved drugs” refers to agents (i.e., chemical entities or biological factors) that have been approved for therapeutic use as drugs in human beings or animals in the United States or other jurisdictions. In the context of the present invention, the term “already-approved drug” means a drug having approval for an indication distinct from an indication being tested for by use of the methods disclosed herein.

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homophenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or (L)-configuration. If non-naturally occurring side chains are used, non-amino acid substituents may be used, for example to prevent or retard in vivo degradations. Peptide inhibitors of enzymes find particular use.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

In some embodiments, the candidate agents are peptides. In this embodiment, it can be useful to use peptide constructs that include a presentation structure. By “presentation structure” or grammatical equivalents herein is meant a sequence, which, when fused to candidate bioactive agents, causes the candidate agents to assume a conformationally restricted form. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present randomized peptide structures. Preferred presentation structures maximize accessibility to the peptide by presenting it on an exterior loop. Accordingly, suitable presentation structures include, but are not limited to, minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc. See See U.S. Pat. No. 6,153,380, incorporated by reference.

Of particular use in screening assays are phage display libraries; see See e.g., U.S. Pat. Nos. 5,223,409; 5,403,484; 5,571,698; and 5,837,500, all of which are expressly incorporated by reference in their entirety for phage display methods and constructs. In general, phage display libraries can utilize synthetic protein (e.g. peptide) inserts, or can utilize genomic, cDNA, etc. digests.

In a preferred embodiment, the candidate agents are antibodies, a class of proteins. The term “antibody” includes full-length as well antibody fragments, as are known in the art, including Fab Fab2, single chain antibodies (Fv for example), chimeric antibodies, humanized and human antibodies, etc., either produced by the modification of whole antibodies or those synthesized de novo using recombinant DNA technologies, and derivatives thereof.

In a preferred embodiment, the candidate bioactive agents are nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook, and peptide nucleic acids. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine, etc.

In one embodiment, the nucleic acids are aptamers, see U.S. Pat. Nos. 5,270,163, 5,475,096, 5,567,588, 5,595,877, 5,637,459, 5,683,867, 5,705,337, and related patents, hereby incorporated by reference.

It should be noted in the context of the invention that nucleosides (ribose plus base) and nucleotides (ribose, base and at least one phosphate) are used interchangeably herein unless otherwise noted.

As described above generally for proteins, nucleic acid candidate bioactive agents may be naturally occurring nucleic acids, random and/or synthetic nucleic acids. For example, digests of procaryotic or eucaryotic genomes may be used as is outlined above for proteins. In addition, RNAis are included herein.

“Food additive” includes, but is not limited to, organoleptic agents (i.e., those agents conferring flavor, texture, aroma, and color), preservatives such as nitrosamines, nitrosamides, N-nitroso substances and the like, congealants, emulsifiers, dispersants, fumigants, humectants, oxidizing and reducing agents, propellants, sequestrants, solvents, surface-acting agents, surface-finishing agents, synergists, pesticides, chlorinated organic compounds, any chemical ingested by a food animal or taken up by a food plant, and any chemical leaching into (or otherwise finding its way into) food or drink from packaging material. The term is meant to encompass those chemicals which are added into food or drink products at some step in the manufacturing and packaging process, or find their way into food by ingestion by food animals or uptake by food plants, or through microbial byproducts such as endotoxins and exotoxins (pre-formed toxins such as botulinin toxin or aflatoxin), or through the cooking process (such as heterocyclic amines, e.g., 2-amino-3-methyllimidazo[4,5-f]quinolone), or by leaching or some other process from packaging material during manufacturing, packaging, storage, and handling activities.

“Industrial chemical” includes, but is not limited to, volatile organic compounds, semi-volatile organic compounds, cleaners, solvents, thinners, mixers, metallic compounds, metals, organometals, metalloids, substituted and non-substituted aliphatic and acyclic hydrocarbons such as hexane, substituted and non-substituted aromatic hydrocarbons such as benzene and styrene, halogenated hydrocarbons such as vinyl chloride, aminoderivatives and nitroderivatives such as nitrobenzene, glycols and derivatives such as propylene glycol, ketones such as cyclohexanone, aldehydes such as furfural, amides and anhydrides such as acrylamide, phenols, cyanides and nitriles, isocyanates, and pesticides, herbicides, rodenticides, and fungicides.

“Environmental pollutant” includes any chemical not found in nature or chemicals that are found in nature but artificially concentrated to levels exceeding those found in nature (at least found in accessible media in nature). So, for example, environmental pollutants can include any of the non-natural chemicals identified as an occupational or industrial chemical yet found in a non-occupational or industrial setting such as a park, school, or playground. Alternatively, environmental pollutants may comprise naturally occurring chemicals such as lead but at levels exceeding background (for example, lead found in the soil along highways deposited by the exhaust from the burning of leaded gasoline in automobiles). Environmental pollutants may be from a point source such as a factory smokestack or industrial liquid discharge into surface or groundwater, or from a non-point source such as the exhaust from cars traveling along a highway, the diesel exhaust (and all that it contains) from buses traveling along city streets, or pesticides deposited in soil from airborne dust originating in farmlands. As used herein, “environmental contaminant” is synonymous with “environmental pollutant.”

In general, libraries of candidate agents are tested. By “library” herein is meant a plurality of molecules, and in general is at least 10² to 10⁸ molecules, with from about 10² to about 10⁴ to 10⁶ being preferred.

The candidate agents are added to cells for evaluation. As will be appreciated by those in the art, this can be done in a wide variety of ways. In general, the candidate agents are added to the cell media and the mixture is allowed to incubate for some period of time. Uptake agents may be used, such as liposomes, etc., or electroporation or other methods of introducing the candidate agents to the cells. In general, the addition step will be done under physiological conditions suitable for cell growth.

### Suitable Cell Types

Depending on the assay and desired outcome, a wide variety of cell types may be used, including eukaryotic and prokaryotic cells.

Suitable eukaryotic cells include any animal, plant, yeast, fungal and protozoa cells. In some aspects, preferred eukaryotic cells are mammalian cells, with rodent (e.g. mouse, rat, hamster, etc.) primate and human cells being particularly preferred. Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, adipocytes, pancreatic cells, islets of Langheran, neural cells (including immortalized neuoendocrine cells, neuroblastoma cells and glia cells), Schwanoma cell lines, organotypic or mixed cells in culture and beta cells . . . . Suitable cells also include known research cells, including, but not limited to, Jurkat T cells, NIH 3T3 cells, CHO, COS, etc. See the ATCC cell line catalog, hereby expressly incorporated by reference.

Preferred eukaryotic cells include yeast cells, particularly Saccharomyces cerevisiae, Hansenula polymorpha, Kluyveromyces fragilis and K. lactis, Pichia guillerimondii and P. pastoris, Schizosaccharomyces pombe, plasmodium falciparium, and Yarrowia lipolytica.

Additional preferred fungal cells include, but are not limited to, Candida strains including Candida glabrata, Candida albicans, C. krusei, C. lusitaniae and C. maltosa, as well as species of Aspergillus, Cryptococcus, Histoplasma, Coccidioides, Blastomyces, Penicillium.

Preferred protozoa include, but are not limited to, Trypanosoma, Leishmania species including Leishmania donovanii, Plasmodium spp., Pneumocystis carinii, Cryptosporidium parvum, Giardia lamblia, Entamoeba histolytica, and Cyclospora cayetanensis.

In one embodiment, the cells utilized in the assays are prokaryotic, for example to test for modulation of infectivity and activity based on metalation. For example, for some bacteria, virulence is associated with metal uptake, for example iron uptake. Thus, testing for modulation (e.g. inhibition) of metalation of bacterial proteins is provided. Suitable bacteria include, but are not limited to, pathogenic and non-pathogenic prokaryotes including Bacillus, including Bacillus anthracis; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. Y. lamblia, Y. pestis, Pseudomonas, e.g. P. aeruginosa, P. putida; Chlamydia, e.g. C. trachomatis; Bordetella, e.g. B. pertussis; Treponema, e.g. T. palladium; B. anthracis, Y. pestis, Brucella spp., F. tularensis, B. mallei, B. pseudomallei, B. mallei, B. pseudomallei, C. botulinum, Salmonella spp., SEB V. cholerae toxin B, E. coli O157:H7, Listeria spp., Trichosporon beigelii, Rhodotorula species, Hansenula anomala, Enterobacter sp., Klebsiella sp., Listeria sp., Mycoplasma sp. and the like.

In one embodiment, the cells may be genetically engineered, for example they may contain exogenous nucleic acids or have endogenous nucleic acids removed (e.g. “knock outs”). As is more fully described below, this may be to include metalation proteins in cell lines (e.g. the addition of human metal ion membrane pumps to yeast cells that have had endogeneous metal pumps disrupted or removed), to include components for yeast two hybrid systems, the use of fusion partners on target proteins to facilitate purification, etc.

In some aspects, the cells may be infected with viruses to test for agents that modulate viral infection and activity based on metalation. These viruses include, but are not limited to, including orthomyxoviruses, (e.g. influenza virus), paramyxoviruses (e.g. respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like. In this embodiment, it is possible to add the candidate agent prior to, simultaneously with, or after the introduction of the virus. The infection efficiency and the subsequent ability of the virus to transfect, replicate, undergo release and subsequent reinfection cycles can all be scored by standard methods.

The candidate agents are added to the cells and allowed to incubate for a suitable period of time. In one embodiment, the metalation status of the target metalloprotein is evaluated. By “metalloprotein” herein is meant a protein that reversibly binds at least one metal ion. The metal ion is bound by one or more side chain or backbone atoms or by other binding functions that are bound to the protein; metalloproteins exhibit characteristic metal-to-protein stoichiometries that correspond to the minimal metal-protein ratio for a given structure or function; the structure and/or function of this type of protein may be dependent upon the metal being bound in a specific site of the protein. If metal-occupancy is required for a specific catalytic activity, the metalloproteins is called a metalloenzyme or metal-activated protein. It should be noted that the term “mp” includes the apoprotein forms as well; that is, metalloproteins may be metalated or not, as is described herein.

In general, the metalloproteins of the invention are target metalloproteins. By “target metalloproteins” herein is meant a particular metalloprotein for which modulators of activity are desired, due to their role in cellular function. Suitable target metalloproteins are outlined below.

As indicated herein, the metal ion may directly contribute to bioactivity, such as in the case of copper oxidases, heme proteins or MMPs, function to confer structural integrity, such as the case in zinc finger transcription factors, function in a regulatory manner, such as in the case of metalloregulatory proteins, or function in metal trafficking, such as metallochaperone proteins, to allow bioactivity. “Bioactivity” in this context includes both enzymatic activity or activity based on protein binding (e.g. transcription factors), and will be determined based on the identity of the metalloprotein. There are a wide number of suitable metalloproteins for use in the invention. In one embodiment, the metalloprotein is an enzyme, sometimes referred to herein as a metalloenzyme. Suitable classes of metalloenzymes include, but are not limited to, hydrolases such as proteases, carbohydrases, phosophotases, lipases and nucleases; oxidoreductases such as oxygenases, oxidases, reductases; electron transfer proteins; isomerases such as racemases, epimerases, tautomerases, or mutases; transferases, kinases and phophatases.

In some embodiments, the metalloproteins are not enzymes, but have bioactivity based on other protein characteristics. Zn finger proteins are an example as are, metal trafficking proteins such as metallochaperones, metal transport proteins, and metalloregulatory proteins, which are metal sensors that control gene expression or signal transduction pathways.

Particular metalloproteins of use in the invention include, but are not limited to, matrix metalloproteinases (including MT-MMPs) such as MMP-1, MMP-2, MMP-3, MMP-4, MMP-5, MMP-6, MMP-7, MMP-8, MMP-9, MMP-10, MMP-11, MMP-12, MMP-13, MMP-14, MMP-15, MMP-16 and MMP-17, myelin basic protein (MBP), NS5A of HCV, cytochrome c oxidase, VAP-1, SOD, anthrax lethal factor, 5-lipoxygenase, COX-2, VAP-1, PDF, 4-hydroxyphenylpyruvate dioxygenase, alcohol dehydrogenase, angiotensin converting enzyme (ACE), aromatase, metallo-beta-lactamase (bacterial), carbamoyl phosphate synthetase, carbonic anhydrase II, carbonic anhydrase I, catechol o-methyl-transferase, cyclooxygenase (non-selective), cyclooxygenase 2, cytochrome P450, DNA polymerase, EGFR tyrosine kinase, farnesyl diphosphate synthase, fumarate reductase (mitochondrial), heme oxygenase, HIV-1 reverse transcriptase, HIV-1 reverse transcriptase, lanosterol demethylase (fungal), neutral endopeptidase, PDE (non-selective), PDE III selective, PDE IV selective, PDE V selective, pyruvate:ferredoxin oxidoreductase, renal dipeptidase, ribonucleoside diphosphate reductase, RNA polymerase (bacterial), thyroid peroxidase, and xanthine oxidase. Iron dependent proteins include, but are not limited to, heme proteins including COX-2, alpha keto glutarate dependent enzymes which require a non-heme iron cofactor; ribonucleotide reductase (many forms have two non-heme irons in the active site), peptide deformylase, anthrax lethal factor, etc.

As outlined herein, metalloproteins contain one or more metal ions, including, but not limited to, Scandium, Titanium, Vanadium, Chromium, Manganese, Iron, Cobalt, Nickel, Copper, Zinc, Yttrium, Zirconium, Niobium, Molybdenum, Technetium, Ruthenium, Rhodium, Palladium, Silver, Cadmium, Hafnium, Tantalum, Tungsten, Rhenium, Osmium, Iridium, Platinum, Gold, Mercury,

### Metalation Status

Once the candidate agent and the cells have been incubated if required, in some assays of the invention, the metalation status of the metalloprotein is determined as a readout of the ability of the candidate agent to modulate metalation. By “metalation status” herein is meant the presence or absence of one or more metal ions associated with the target metalloprotein; that is, whether the metalloprotein is in an apoprotein form. By “apoprotein” herein is meant a metalloprotein that has at least one functional metal ion absent or removed. In this context, “functional metal ion” is a metal ion whose absence from the metalloprotein causes either a decrease or elimination of activity. In the case where the metalloprotein has more than one functional metal ion, some embodiments measure partially unmetalated proteins, as long as the loss of some measurable bioactivity is seen with the partially unmetalated protein. In the case where the metalloprotein contains two or more different functional metal ions, “apoprotein” includes the loss of one type of metal ion but not the other, as well as the loss of both. In general, the methods of the invention utilize fully unmetalated proteins. It should be noted that in some cases, a complete lack of metalation is not required; for example, even if some of the proteins in the sample retain metal, and thus activity, as long as a decrease in activity is measurable as additional functional metals are removed, the sample is appropriate for use in the methods outlined herein.

In general, metalation status is determined by assaying for bioactivity of the particular target metalloprotein. This is generally done by isolating or purifying the metalloprotein after the assay is completed. Metalloproteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the metalloprotein may be purified using a standard anti-metalloprotein antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the metalloprotein. In some instances no purification will be necessary.

In addition, in some embodiments, gross cellular fractionation with other techniques will be sufficient to allow the determination of metalation status. For example, elemental analysis, SDS PAGE gels and mass spectroscopy techniques can allow the determination of the presence of metal in certain fractions.

Once purified, a variety of methods to determine metalation status can be used. A combination of atomic absorption, protein hydrolysis and mass spectroscopy techniques including MALDI-TOF and ICPMS allows the determination of the specific metal cofactors and further establishes the metal-protein ratio in the purified proteins. These methods are used to calibrate more convenient spectroscopic methods for monitoring changes in metal occupancy in subsequent screening assays. These include, but are not limited to, spectroscopic changes in the protein or metal-based chromaphores and fluorophores. The spectroscopic methods include UV-VIS and other optical techniques, fluorescence emission, fluorescence anisotropy, electron paramagnetic resonance and mass spectroscopy techniques. In other assays, metal binding dyes whose optical or fluorescence properties increase or decrease with the addition or loss of metal from the purified protein can be used; bioactivity assays based on the biochemical activity of the particular metalloprotein,

In one embodiment, the purified metalloprotein (which may or may not be fully or partially unmetalated) is fractionated, characterized and the metal content calibrated as described above to determine the presence or absence of metal ion in the metalloprotein.

In one embodiment, the purified metalloprotein is tested optically; for example, some metalloproteins alter their absorbance spectra upon addition or loss of the metal. For example, some proteins are different spectroscopic changes (for instance colors) depending on the presence or absence of a metal ion. Similarly, conformation changes can be monitored using UV/Visible absorbance or fluorescence. For example, the lack of structural metal ions can “loosen” the conformation structure such that the absorbance or fluoresence of tyrptophan and tyrosine are altered, relative to the structure with metal present.

In some cases, dyes can be used to determine metalation status by competition methods. For example, for Zn metalloproteins, the many types of metal-specific fluorescent probes, such as those in the Zinbo and Zinquin families can be used. These probes are fluorescent dyes coupled to Zn chelators that alter their absorption or emission spectra upon Zn binding (see Taki et al, JACS 126:712 (2994) and Fahrni et al., JACS 121:11448 (1999), both of which are hereby incorporated by reference in their entirety). If a candidate agent blocks Zn-acquisition by a target protein in the assay after a given incubation time, then the subsequent aliquot of the Zn-probe complex will not undergo a change in fluorescence. In control assays, those compounds that do not block acquisition of Zn the target protein will allow the target apo-mp to take Zn from the Zn-probe complex and a change in the fluorescence of the sample will be observed.

Thus, the present invention is directed in some embodiments to methods of identifying modulators of metalation. In this context, “modulation” includes both activation and inhibition, with the latter generally preferred. In some cases, modulation includes a change in the presence of metal in the target metalloprotein, and generally a corresponding modulation in the bioactivity of the target metalloprotein. In general, changes of at least 5-25% are preferred, with changes of greater than 50% particularly preferred.

### Altered Phenotype Readout

In addition or instead of the evaluation of metalation status, in some embodiments, the cells are evaluated for altered phenotypes.

By “altered phenotype” or “changed physiology” or other grammatical equivalents herein is meant that the phenotype of the cell is altered in some way, preferably in some detectable and/or measurable way. As will be appreciated in the art, a strength of the present invention is the wide variety of cell types and potential phenotypic changes which may be tested using the present methods. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density; changes in the expression of one or more RNAs, proteins, lipids, hormones, cytokines, or other molecules; changes in the equilibrium state (i.e. half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytokines, receptors, or other molecules; changes in the secretion of ions, cytokines, hormones, growth factors, or other molecules; alterations in cellular membrane potentials, polarization, integrity or transport; changes in infectivity, susceptability, latency, adhesion, and uptake of viruses and bacterial pathogens; etc. By “capable of altering the phenotype” herein is meant that the bioactive agent can change the phenotype of the cell in some detectable and/or measurable way.

The altered phenotype may be detected in a wide variety of ways, as is described more fully below, and will generally depend and correspond to the phenotype that is being changed. Generally, the changed phenotype is detected using, for example: microscopic analysis of cell morphology; standard cell viability assays, including both increased cell death and increased cell viability, for example, cells that are now resistant to cell death via virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; etc.

Once an altered phenotype has been detected, a variety of additional assays may be done. In one embodiment, the metalation status of a target protein is done as outlined above.

### Screening for Inhibitors of Metalation Pathway Proteins

In addition to cell based screening, screening for modulators of particular metalation pathway proteins can be done as well.

In this embodiment, known metalation pathway proteins are tested. In general, pathway proteins fall into several distinct categories, including, but not limited to, integral transmembrane transporters (sometimes referred to herein as “metal ion pumps”), metalloregulatory sensor proteins, protein folding assemblies, proteins that act as a scaffold for assembly of iron-sulfur cofactors, and soluble or membrane bound metallochaperone proteins.

It should be noted that many of the pathway proteins are themselves metalloproteins by definition. For example, in the case of metallochaperones, the activity is the transport of metal ions; as such, activity is dependent on the presence of the metal, and thus falls within the definition of a metalloprotein as a protein that reversibly binds at least one metal ion. As such, the metallochaperones have a metalated and unmetalated (apo) form. Similarly, many of the metalloregulatory sensor proteins rely on the presence or absence of metal ions to trigger activity.

In one embodiment, screening of metallochaperone proteins is done. Suitable putative metallochaperone proteins include, but are not limited to, Fe chaperones (including HFE, Frataxin, CcmE and homologs and orthologs thereof) Cu chaperones (including Atx1, Hah1 (also known as Atox1), CCS, Cox17, Sco1, Sco2, Cox19, CopZ, CusF, CopC, PcoC, PcoE and homologs and orthologs thereof)) Zn chaperones (including metallothionein, calreticulin, YodA/ZinT, HSP33 the N-terminal domains of ZntA and CCS), Ni chaperones (UreE and homologs and orthologs). In some cases, metallochaperones are particularly useful as targets for modulation of activity, as many of them utilize non-standard chelation chemistry to transfer metal ions to their ultimate target metalloprotein. As outlined in co-pending U.S. Ser. No. 60/728,840 titled “Compositions and Methods of Screening Apoproteins”, filed Oct. 20, 2005, hereby incorporated by reference in its entirety and for screening techniques, the metallochaperones can be screened as apoproteins, e.g. in an unmetalated state, to find inhibitors of metalation of the metallochaperone. Alternatively, the assay readout can be based on the lack of metalation of the target metalloprotein; thus, for example, in an assay for inhibition of a Cu metallochaperone protein, the readout is the metalation and/or activity of the corresponding metalloprotein that normally receives its metal from the metallochaperone protein. Again, this may be done as outlined herein by detecting the metalation status either directly (e.g. looking for the presence or absence of metal, for example using mass spectroscopy) or via bioactivity assays depending on the identity of the metalloprotein.

In one embodiment, screening of metalloregulatory sensor proteins is done. For example, E. coli proteins Zur and ZntR regulate the transcription of Zn pumps, and homologs and orthologs of these proteins are included. Again, the assays can be done as outlined herein on the basis of the presence or absence of metal in the metalloregulatory protein or on the basis of the activated gene or protein (e.g. determining the presence of mRNA, for example, or of the protein itself).

In one embodiment, screening of integral transmembrane transporter proteins and their partners (sometimes referred to herein as “metal ion pumps”) is done. For example, E. coli proteins ZnuABC are part of the ABC transport family which includes a separate Zn-binding protein (ZnuA), a membrane bound Zn-permease (ZnuB) and an ATPase (ZnuC). Another transport system has one protein, ZntA is a zinc utilizing P-type ATPase: it is one protein that carries out the zinc pumping function. Other examples include, ZIP proteins (Zrt-, Irt-like proteins also know as SLC39 family including ZupT) and the CDF family (including (ComA (Cu) and homologs and orthologs are included as well. Parallel pathway systems for copper transport are suitable including the ATPase 7a and 7b which encode the Menkes and Wilson Disease proteins, ccc2 and CopA which encode homologs and orthologs, and other, and non-membrane auxiliary proteins such as the multicopper oxidases CueO and PcoA proteins. cusRs, Manganese and iron transport pathway proteins such as the SLC11 family of transporters (which include DCT1/DMT1/Nramp2 proteins) and all manner of siderophore biosynthetic and transport proteins are appropriate as well.

See metalloproteins and metalation pathway proteins listed in Tottey et al., Acc. Chem. Res. 38:775 (2005); Hantke, Curr. Opin. Microbio. 8:196 (2005); Palmiter et al., Eur. J. Physiol. 447:744 (2004); Schapiro et al., PNAS 100:8496 (2003); Furukawa et al., EMBO J. 23(10):2872 (2004); Brown et al., PNAS 101(15):5518 (2004); Changela et al., Science 301:1383 (2003); Finney et al., Science 300:931 (2003); Wommer et al., J. Biol. Chem. 277:24142 (2002); Arnesano et al., J. Biol. Chem. 279:47998 (2004); Outten et al., Science 292:2488 (2001); and Science 1993 Aug. 6; 261(5122):699-700; all of which are expressly incorporated by reference in their entirety, particularly for metalloproteins and pathway proteins.

In some embodiments, the pathway proteins are variants, including amino acid substitutions and deletions, such as truncations. For example, due to the general difficulty of handling transmembrane proteins, either the intracellular domain, the extracellular domain, or the metal binding regions, depending on the activity to be modulated, may be used (e.g. for transporter proteins, the transmembrane domain may be deleted).

In general, synthetic peptides or recombinant techniques can be used to generate the pathway proteins or the relevant domains, as outlined below. In general, pathway protein genes are isolated as is known in the art, generally by using primers and amplification systems such as polymerase chain reaction (PCR) from known sequences such as those found in GenBank. A variety of expression vectors can be made. The expression vectors may be either self-replicating extrachromosomal vectors or vectors which integrate into a host genome. Generally, these expression vectors include transcriptional and translational regulatory nucleic acid operably linked to the nucleic acid encoding the pathway protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the metalloprotein; for example, transcriptional and translational regulatory nucleic acid sequences from Bacillus are preferably used to express the metalloprotein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The metalloproteins of the present invention are produced by culturing a host cell transformed with an expression vector containing nucleic acid encoding a metalloprotein, under the appropriate conditions to induce or cause expression of the metalloprotein. The conditions appropriate for metalloprotein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archebacteria, fungi, and insect and animal cells, including mammalian cells. Of particular interest are Drosophila melangaster cells, Saccharomyces cerevisiae and other yeasts, E. coli strains, Bacillus subtilis, SF9 cells, C129 cells, 293 cells, Neurospora, BHK, CHO, COS, and HeLa cells, fibroblasts, immortalized neuoendocrine cells, neuroblastoma cells, glia cells, pancreatic cells, Schwanoma cell lines, organotypic or mixed cells in culture and other immortalized mammalian cell lines.

In a preferred embodiment, the metalloproteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for Metalloprotein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, metalloproteins are expressed in bacterial systems. Bacterial expression systems are well known in the art. A suitable bacterial promoter is any nucleic acid sequence capable of binding bacterial RNA polymerase and initiating the downstream (3′) transcription of the coding sequence of the metalloprotein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the metalloprotein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatment, electroporation, and others.

In one embodiment, proteins are produced in insect cells. Expression vectors for the transformation of insect cells, and in particular, baculovirus-based expression vectors, are well known in the art.

In a preferred embodiment, the protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

In a preferred embodiment, the protein is purified or isolated after expression, as outlined above.

Once expressed and purified if necessary, the pathway proteins are useful in a number of applications, and in particular, screening assays for the identification of candidate agents that bind to the pathway proteins and prevent metalation of target metalloproteins, such that bioactivity is decreased.

Screens may be designed to first find candidate agents that can bind to pathway proteins, and then these agents may be used in assays that evaluate the ability of the candidate agent to modulate bioactivity. Thus, as will be appreciated by those in the art, there are a number of different assays which may be run; binding assays and activity assays.

Thus, in a preferred embodiment, the methods comprise combining a pathway protein and a candidate bioactive agent, and determining the binding of the candidate agent to the pathway protein.

### Assay Components

The candidate agents are contacted with the pathway protein under reaction conditions that favor agent-target interactions. Generally, this will be physiological conditions. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away, in the case of solid phase assays. Assay formats are discussed below.

A variety of other reagents may be included in the assays. These include reagents like salts, neutral proteins, e.g. albumin, detergents, etc which may be used to facilitate optimal apoprotein-agent binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

It should be noted that in most cases, the assays will be run in the presence of metal ions. For screening of pathway proteins that are themselves metalloproteins by utilizing the apoprotein form, see U.S. Ser. No. 60/728,840 filed concurrently with the present application on Oct. 20, 2005.

In one embodiment, solution phase binding assays are done. Generally in this embodiment, fluorescence resonance energy transfer (FRET) assays are done, by labeling both the candidate agents and the pathway proteins with different fluorophores with overlapping spectra. As energy transfer is distance dependent, in the absence of binding the excitation at one wavelength does not produce an emission spectra. Only if the two labels are close, e.g. when binding has occurred, will excitation at one wavelength result in the desired emission spectra of the second label.

In some embodiments, solid phase (heterogeneous) assays are done. In this case, binding assays are done wherein either the pathway protein or the candidate agent is non-diffusably bound to an insoluble solid support, and detection is done by adding the other component which is labeled, as described below. The insoluble supports may be made of any composition to which the compositions can be bound, is readily separated from soluble material, and is otherwise compatible with the overall method of screening. The surface of such supports may be solid or porous and of any convenient shape. Examples of suitable supports include microtiter plates, arrays, membranes and beads, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica based materials including silicon and modified silicon, carbon, metals, inorganic glasses, plastics, ceramics, and a variety of other polymers. In a some embodiments, the solid supports allow optical detection and do not themselves appreciably fluoresce. In addition, as is known the art, the solid support may be coated with any number of materials, including polymers, such as dextrans, acrylamides, gelatins, agarose, etc. Exemplary solid supports include silicon, glass, polystyrene and other plastics and acrylics. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable.

In a preferred embodiment, the pathway protein is bound to the support, and a library of candidate bioactive agents are added to the assay. Alternatively, the candidate agent is bound to the support and the pathway protein is added. Attachment to the solid support is accomplished using well known methods, and will depend on the composition of the two materials to be attached. In general, for covalent attachment, attachment linkers are utilized through the use of functional groups on each component that can then be used for attachment. Preferred functional groups for attachment are amino groups, carboxy groups, oxo groups, hydroxyl groups and thiol groups. These functional groups can then be attached, either directly or indirectly through the use of a linker. Linkers are well known in the art; for example, homo- or hetero-bifunctional linkers as are well known (see 1994 Pierce Chemical Company catalog, technical section on cross-linkers, pages 155-200, incorporated herein by reference). In some embodiments, absorption or ionic interactions are utilized. In some cases, small molecule candidate agents are synthesized directly on microspheres, for example, which can then be used in the assays of the invention.

Following binding of the protein or agent, excess unbound material is removed by washing. The surface may then be blocked through incubation with bovine serum albumin (BSA), casein or other innocuous protein or other moiety.

In the binding assays, either the pathway protein, the candidate agent or a reagent that can release the metal ion to the pathway protein (ie the metal donor), is labeled. By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal. Specific labels include optical dyes, including, but not limited to, chromophores, phosphors and fluorophores, with the latter being specific in many instances. Fluorophores can be either “small molecule” fluores, or proteinaceous fluores as described herein. The labeled metal donor can be chemical probe (such as Zinquin or Zinbo5) which undergoes a spectroscopic change when it releases the metal ion as described previously.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes and metal probes, including fluorophores, are described in Molecular Probes Handbook by Richard P. Haugland, hereby expressly incorporated by reference.

In some embodiments, the pathway proteins are fusion proteins. For example, pathway proteins may be modified in a way to form chimeric molecules comprising a pathway protein fused to another, heterologous polypeptide or amino acid sequence. In one embodiment, such a chimeric molecule comprises a fusion of a pathway protein with a tag polypeptide which provides an epitope to which an anti-tag antibody can selectively bind. The epitope tag is generally placed at the amino- or carboxyl-terminus of the pathway protein. The presence of such epitope-tagged forms of a pathway protein can be detected using an antibody against the tag polypeptide. Also, provision of the epitope tag enables the pathway protein polypeptide to be readily purified by affinity purification using an anti-tag antibody or another type of affinity matrix that binds to the epitope tag. These epitope tags can be used for immobilization to a solid support, as outlined herein.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Other suitable fusion partners include other immobilization components, such as histidine tags for attachment to surfaces with nickel, functional components for the attachment of linkers and labels, etc., and proteinaceous labels. In general, attachment will generally be done as is known in the art, and will depend on the composition of the two materials to be attached. In general, attachment linkers are utilized through the use of functional groups on each component that can then be used for attachment. Preferred functional groups for attachment are amino groups, carboxy groups, oxo groups, hydroxyl groups and thiol groups. These functional groups can then be attached, either directly or indirectly through the use of a linker. Linkers are well known in the art; for example, homo- or hetero-bifunctional linkers as are well known (see 1994 Pierce Chemical Company catalog, technical section on cross-linkers, pages 155-200, incorporated herein by reference). Preferred attachment linkers include, but are not limited to, alkyl groups (including substituted alkyl groups and alkyl groups containing heteroatom moieties), with short alkyl groups, esters, amide, amine, epoxy groups and ethylene glycol and derivatives being preferred.

In one embodiment, particularly when the candidate agents are immobilized on a solid support, a suitable fusion partner is an autofluorescent protein label. Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

In one embodiment, the pathway protein is attached to the support, adding labeled candidate agents, washing off excess reagent, and determining whether the label is present on the solid support. Various blocking and washing steps may be utilized as is known in the art.

In one embodiment, the candidate agents are immobilized to the support, and a labeled pathway protein is added to determine binding.

### Robotics

In one embodiment, any of the assays outlined herein can utilize robotic systems for high throughput screening. Many systems are generally directed to the use of 96 (or more) well microtiter plates, but as will be appreciated by those in the art, any number of different plates or configurations may be used. In addition, any or all of the steps outlined herein may be automated; thus, for example, the systems may be completely or partially automated.

As will be appreciated by those in the art, there are a wide variety of components which may be used, including, but not limited to, one or more robotic arms; plate handlers for the positioning of microplates; automated lid handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, tubes, magnetic particle, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, electroporator, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of the heat exchangers such as controlled blocks or platforms to provide accurate temperature control of incubating samples from 4° C. to 100° C.

In some preferred embodiments, the instrumentation will include a detector, which may be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluoroescence resonance energy transfer (FRET), SPR systems, luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

These instruments can fit in a sterile laminar flow or fume hood, or are enclosed, self-contained systems as needed. Flow cytometry or capillary electrophoresis formats may be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic workstation includes one or more heating or cooling components. Depending on the reactions and reagents, either cooling or heating may be required, which may be done using any number of known heating and cooling systems, including Peltier systems.

In a preferred embodiment, the robotic apparatus includes a central processing unit that communicates with a memory and a set of input/output devices (e.g., keyboard, mouse, monitor, printer, etc.) through a bus. The general interaction between a central processing unit, a memory, input/output devices, and a bus is known in the art. Thus, a variety of different procedures, depending on the experiments to be run, are stored in the CPU memory.

### Identification of Metalation Pathway Proteins

In one aspect, the invention provides for the identification of additional metalation pathway proteins. In general, this is done using any of a variety of known protein-protein interaction methods.

### Two Hybrid Systems

### Yeast Two Hybrid Systems

The basic yeast two hybrid system requires a protein-protein interaction in order to turn on transcription of a reporter gene. Subsequent work was done in mammalian cells. See Fields et al., Nature 340:245 (1989); Vasavada et al., PNAS USA 88:10686 (1991); Fearon et al., PNAS USA 89:7958 (1992); Dang et al., Mol. Cell. Biol. 11:954 (1991); Chien et al., PNAS USA 88:9578 (1991); and U.S. Pat. Nos. 5,283,173, 5,667,973, 5,468,614, 5,525,490, and 5,637,463, Bartel and Fields, eds., The Yeast Two-Hybrid System, Oxford University Press, New York, N.Y., 1997 all of which are expressly incorporated by reference in their entirety.

The yeast two-hybrid technique is based on the fact that the DNA-binding domain and the transcriptional activation domain of a transcriptional activator contained in different fusion proteins can still activate gene transcription when they are brought into proximity to each other. As shown in FIG. 1, in a yeast two-hybrid system, two fusion proteins are expressed in yeast cells. One has a DNA-binding domain of a transcriptional activator fused to a bait protein. The other, on the other hand, includes a transcriptional activating domain of the transcriptional activator fused to another test protein (or vice versa). If the two test proteins interact with each other in vivo, the two domains of the transcriptional activator are brought together reconstituting the transcriptional activator and activating a reporter gene controlled by the transcriptional activator. See, e.g., U.S. Pat. No. 5,283,173, expressly incorporated by reference in its entirety.

Accordingly, the present invention provides methods and systems for utilizing yeast two-hybrid screens. Thus, in the two-hybrid system, a first protein, or “bait protein”, as termed herein, is fused to a nucleic acid binding domain of a nucleic acid binding protein, such as a transcriptional activator protein, and a second protein, or “test protein”, is fused to the activator domain of a transcriptional activator. If the bait protein and the test protein bind, i.e. have a specific protein-protein interaction, the activator domain is brought into position near the nucleic acid binding domain, and transcription of a detectable gene occurs. If there is little or no interaction, there little or is no detectable protein made.

In a preferred embodiment, either the vector (particularly the test vector) or one or both of the fusion constructs may contain a “rescue” sequence. A rescue sequence is a sequence (either nucleic acid or amino acid) which may be used to purify or isolate either the test or bait proteins or the nucleic acid encoding them. Thus, for example, protein rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST.

Alternatively, the rescue sequence may be a unique oligonucleotide sequence which serves as a probe target site to allow the quick and easy isolation of the retroviral construct, via PCR, related techniques, or hybridization.

In a preferred embodiment, the first fusion gene comprises a first sequence encoding a nucleic acid binding domain, and the second sequence encodes a bait protein. By “nucleic acid binding domain” herein is meant a proteinaceous domain which is able to bind a specific nucleic acid sequence, generally a DNA sequence. As noted above, transcriptional activation proteins generally contain at least two domains, a nucleic acid binding domain and a transcriptional activation domain; for the purposes of the present invention, the nucleic acid binding domain and the transcriptional activation domain may come from the same protein or different proteins. As will be appreciated by those in the art, what is important is that the transcriptional activator from which these sequences are derived have functionally distinct domains. Suitable nucleic acid binding domains include, but are not limited to, nucleic acid binding domains from Tet, GAL4 (amino acids 1-147; Fields et al., supra; see also Gill et al., PNAS USA 87:2127 (1990); Chasman et al., Mol. Cell. Biol. 9:4746 (1989)); LexA (Thliveris et al., Proc. Natl. Acad. Sci. 1992; Hurstels et al., EMBO 1986); GCN4 from S. cerevisiae (Hope et al., Cell 46:885 (1986); ARD1 from S. cerevisiae (Thukral et al., Mol. Cell. Biol. 9:2360 (1989), the human estrogen receptor (Kumar et al., Cell 51:941 (1987), and NF-kB p65, and p53, and derivatives thereof which are functionally similar.

In a preferred embodiment, the first fusion gene further comprises a second sequence encoding a bait protein. “Protein” in this context includes peptides, oligopeptides and proteins. By “bait protein” herein is meant a protein which is to be tested for interaction with another protein. Generally, the bait protein comprises all or part of a target molecule which has either been implicated in a biological process of interest or for which the function is sought. Suitable bait proteins are any metalloprotein or pathway protein as outlined herein, and can include fragments, derivatives and variants thereof.

Customarily one bait protein is used to test a library of test sequences as is described below; however, as will be appreciated by those in the art, the bait protein may be one of a library as well, thus forming an experimental matrix wherein two libraries (although the coding regions of the libraries could be identical) are evaluated for protein-protein interactions. In a preferred embodiment, self-activating bait proteins are filtered out from the bait protein library.

The present invention also provides test vectors. Generally, the test vector is a distinct vector from the bait vector, although as will be appreciated by those in the art, one or more independent vectors may be used. That is, the components of the bait and test vectors could reside on a single vector or on two vectors. Similarly, the reporter vector can be independent or part of either the bait or test vector, or the entire system may reside on a single vector, if the size of the vector is not a concern. Generally, when the test protein is a member of a library, as is outlined below, the test vector will be separate from the bait and reporter vectors.

The test vector also generally comprises a selection gene. Preferably, when the bait and test vectors are distinct, the selection gene of the test vector is different from the selection gene of the bait vector, to ensure that both vectors are maintained within the cell. However, in some embodiments this may not be required; accordingly, the first and second selection genes may be the same or different.

The test vector further comprises a second fusion gene comprising a third sequence encoding a transcriptional activator domain and a fourth sequence encoding a test protein. As above, these may be fused directly or via a linker. By “transcriptional activator domain” herein is meant a proteinaceous domain which is able to activate transcription.

Suitable transcription activator domains include, but are not limited to, transcriptional activator domains from GAL4, GCN4, ARD1, the human estrogen receptor, VP16 (Triezenberg et al., Genes Dev. 2(6):718-729 (1988)), and B42 (Gyuris et al, Cell 1993), and NF-kB p65, and derivatives thereof which are functionally similar.

The fourth sequence encodes a test protein. By “test protein” herein is meant a candidate protein which is to be tested for interaction with a bait protein. Protein in this context means proteins, oligopeptides, and peptides, i.e. at least two amino acids attached. In a preferred embodiment, the test protein sequence is one of a library of test protein sequences; that is, a library of test proteins is tested for binding to one or more bait proteins. The test protein sequences can be derived from genomic DNA, cDNA or can be random sequences. Alternatively, specific classes of test proteins may be tested. The library of test proteins or sequences encoding test proteins are incorporated into a library of test vectors, each or most containing a different test protein sequence.

In a preferred embodiment, the test protein sequences are derived from genomic DNA sequences. Generally, as will be appreciated by those in the art, genomic digests are cloned into test vectors. The genomic library may be a complete library, or it may be fractionated or enriched as will be appreciated by those in the art.

In a preferred embodiment, the test protein sequences are derived from cDNA libraries. A cDNA library from any number of different cells may be used, and cloned into test vectors. As above, the cDNA library may be a complete library, or it may be fractionated or enriched in a number of ways.

The reporter vector further comprises at least one detectable gene, which is transcribed upon activation of the operator, due to a protein-protein interaction of the bait and test proteins. By “detectable gene” herein is meant a gene whose expression results in a detectable phenotype, either by itself or with the addition of a compound or substance which results in a detectable phenotype. Suitable detectable proteins include, but are not limited to, green fluorescent protein and derivatives (see above), luciferase, alkaline phosphatase, chloramphenicol acetyl transferase, lacZ, and drug selection genes (preferably other than those on the bait and test vectors may also be used).

In a preferred embodiment, the expression of the detectable gene allows for cell sorting, such as by fluorescence-activated cell sorting, or FACS.

In a preferred embodiment, once a cell with an altered phenotype is detected, the cell is isolated from the plurality which do not have altered phenotypes. This may be done in any number of ways, as is known in the art, and will in some instances depend on the assay or screen. Suitable isolation techniques include, but are not limited to, drug selection, FACS, lysis selection using complement, cell cloning, scanning by Fluorimager, expression of a “survival” protein, induced expression of a cell surface protein or other molecule that can be rendered fluorescent or taggable for physical isolation; expression of an enzyme that changes a non-fluorescent molecule to a fluoroscent one; overgrowth against a background of no or slow growth; death of cells and isolation of DNA or other cell vitality indicator dyes; changes in fluorescent characteristics, etc. The preferred isolation techniques are drug selection and FACS based on the expression of the detectable gene, with a preferred embodiment utilizing both simultaneously.

Once a cell with a protein-protein interaction is detected and isolated, it is generally desirable to identify the test protein (and the bait protein, if its identity was unknown). In a preferred embodiment, the test protein nucleic acid and/or the test protein is isolated from the positive cell. This may be done in a number of ways. In a preferred embodiment, primers complementary to DNA regions common to the vector, or to specific components of the library such as a rescue sequence, defined above, are used to “rescue” the unique test sequence. Alternatively, the test protein is isolated using a rescue sequence. Thus, for example, rescue sequences comprising epitope tags or purification sequences may be used to pull out the test protein, using immunoprecipitation or affinity columns. In some instances, as is outlined below, this may also pull out the bait protein, if there is a sufficiently strong binding interaction between them. Alternatively, the test protein may be detected using mass spectroscopy.

Once rescued, the sequence of the test protein and/or test nucleic acid is determined. This information can then be used in a number of ways.

In a preferred embodiment, the test protein is resynthesized and reintroduced into the target cells, to verify the effect. This may be done using retroviruses, or alternatively using fusions to the HIV-1 Tat protein, and analogs and related proteins, which allows very high uptake into target cells. See for example, Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); and Baldin et al., EMBO J. 9:1511 (1990), all of which are incorporated by reference.

In a preferred embodiment, either the test protein or the nucleic acid encoding it is used to identify other target molecules, i.e. the initially identified test protein is then used as a bait protein. It is also possible to synthetically prepare labeled proteins and use them to screen a cDNA library expressed in bacteriophage for those cDNAs which bind the protein.

The identified pathway proteins can then be used in any of the methods of the present invention, as well as in U.S. Ser. No. 60/728,840 filed Oct. 20, 2005.

### Mammalian Two Hybrid Systems

In addition to the yeast two hybrid system, several mammalian systems have been identified. See U.S. Pat. Nos. 6,787,321; 6,479,289; 6,251,675; 6,114,111 and 6,316,223, all of which are incorporated by reference.

### Phage Display Systems

In addition to two hybrid systems, a popular approach useful in large-scale screening is the phage display method, in which filamentous bacteriophage particles are made by recombinant DNA technologies to express a peptide or protein of interest fused to a capsid or coat protein of the bacteriophage. A whole library of peptides or proteins of interest can be expressed and a bait protein (e.g. the pathway protein or metalloprotein) can be used to screening the library to identify peptides or proteins capable of binding to the bait protein. See e.g., U.S. Pat. Nos. 5,223,409; 5,403,484; 5,571,698; and 5,837,500, all of which are expressly incorporated by reference in their entirety for phage display methods and constructs.

### Miscellaneous Other Protein-Protein Interaction Assays

Traditional methods for protein-protein interactions are done using biochemical techniques, including, but not limited to, chemical cross-linking, co-immunoprecipitation and co-fractionation and -purification.

### Methods of Inhibiting

In additional embodiments, the invention provides methods of a metalation pathway protein involved in metalating a target metalloprotein comprising contacting a metalation pathway protein with an inhibitor such that metal binding to the metalloprotein is decreased. In this case, “decreased” is generally at least a 5-20-25% decrease in the amount of metal bound per protein, with over 50-75% being useful in some embodiments and a 95-98-100% loss of metal being useful as well. In general, the loss of metal correlates to a loss of biological activity, as measured depending on the metalloprotein, with the same decreases in activity being useful.

In a further embodiment, the invention provides methods of treating a metalloprotein-associated disorder comprising contacting a metalation pathway protein with an inhibitor such that metal binding to the apoprotein form is decreased. In this context, a “metalloprotein-associated disorder” is a disorder either directly or indirectly related to a metalloprotein.

1. A method of screening for alterations in the metalation of a target protein comprising:
a) adding a candidate agent to a cell, wherein the candidate agent comprises an organic compound; and
b) determining the metalation status of said target protein.

2. A method according to claim 1 wherein said metalation status is determined by a bioactivity assay of said protein.

3. A method according to claim 1 wherein said metalation status is determined by purifying said protein and determining the presence or absence of metal ions associated with said protein.

4. A method according to claim 1 wherein said target protein is associated with a cellular phenotype and said metalation status is determined by evaluating said cellular phenotype.

5. A method according to claim 4 wherein said cellular phenotype is evaluated using fluorescence activated cell sorting (FACS).

6. A method according to claim 1 wherein a library of candidate agents are added to a plurality of cells.

7. A method according to claim 1 wherein said candidate agents are labeled.

8. A method of screening for modulation of target metalation pathway proteins comprising:
a) contacting a library of candidate agents with a target pathway protein, wherein the candidate agents comprise an organic compound; and
b) determining the effect of said agent on the activity of said protein.

9. A method of inhibiting a metalation pathway protein involved in metalating a target metalloprotein comprising contacting a metalation pathway protein with an inhibitor such that metal binding to said metalloprotein is decreased.

10. A method according to claim 9 wherein said metalation pathway protein is a metallochaperone protein.

11. A method according to claim 9 wherein said metalation pathway protein is a metal transporter protein.

12. A method according to claim 9 wherein said metalation pathway protein is a metalloregulatory protein.

13. A method of treating a metalloprotein-associated disorder comprising contacting a metalation pathway protein with an inhibitor such that metal binding to said metalloprotein is decreased.

14. A method according to claim 13 wherein said metalation pathway protein is a metallochaperone protein.

15. A method according to claim 13 wherein said metalation pathway protein is a metal transporter protein.

16. A method according to claim 13 wherein said metalation pathway protein is a metalloregulatory protein.
